(19) United States
(12) Patent Application Publication
(10) Pub. No.: US 2012/0253195 A1
(43) Pub. Date: Oct. 4, 2012

(54) ULTRASONIC DIAGNOSTIC APPARATUS, ELASTIC IMAGE STORAGE/REPRODUCTION METHOD, AND ELASTIC IMAGE STORAGE/REPRODUCTION PROGRAM

(75) Inventors: Shinsuke Inoue, Tokyo (JP); Osamu Mori, Tokyo (JP); Koji Waki, Tokyo (JP)

(73) Assignee: HITACHI MEDICAL CORPORATION, Tokyo (JP)

(21) Appl. No.: 13/386,122

(22) PCT Filed: Jul. 20, 2010
(86) PCT No.: PCT/JP2010/062135

§ 371 Date: Jan. 20, 2012

(30) Foreign Application Priority Data
Jul. 24, 2009 (JP) 2009-172747

(51) Int. Cl. A61B 8/08

(52) U.S. Cl. 600/438

(57) ABSTRACT

An ultrasonic diagnostic apparatus of the present invention includes: an ultrasonic probe which transmits and receives an ultrasonic wave to and from an object; a phasing addition means that generates RF signal frame data of a sectional part of the object on the basis of a reflected echo signal measured by the ultrasonic probe; a displacement measurement means that generates displacement frame data by measuring displacement of tissue of the sectional part on the basis of a pair of RF signal frame data items acquired at different times; an elastic information calculation means that generates elastic frame data by calculating elastic information, which indicates hardness or softness of tissue of the sectional part, on the basis of the generated displacement frame data; an elastic image configuration means that generates an elastic image on the basis of the elastic frame data; an image display device which displays the elastic image; and a memory in which the elastic image is stored.

### TECHNICAL FIELD

The present invention relates to an ultrasonic diagnostic apparatus, an elastic image storage/reproduction method, and an elastic image storage/reproduction program and in particular, to a technique for improving the usability for an examiner when an elastic image showing the hardness or softness of tissue of a sectional part of an object is stored in a memory and reproduced.

### BACKGROUND ART

The ultrasonic diagnostic apparatus transmits an ultrasonic wave from an ultrasonic probe into the object, receives from the inside of the object a reflected echo signal of the ultrasonic wave corresponding to the structure of body tissue, forms a tomographic image, for example, a B-mode image, and displays the formed tomographic image for diagnosis.

In recent years, an elastic image showing the hardness or softness of body tissue has been generated by measuring an ultrasonic reception signal (RF signal) while pressing an object with an ultrasonic probe using a manual or mechanical method. That is, displacement of each part of the body occurring due to pressure is calculated on the basis of frame data of two ultrasonic reception signals (RF signal) measured at different times, elastic information such as distortion or an elastic modulus is calculated on the basis of the displacement frame data, and an elastic image is generated and displayed on the basis of the elastic frame data.

For example, as disclosed in PTL 1, an elastic image is displayed by giving color information including red and blue according to distortion or an elastic modulus of body tissue. By displaying mostly the hard part of body tissue, it is possible to easily diagnose the spread or size of a tumor.

On the other hand, for example, as disclosed in PTL 2 and PTL 3, evaluating the display value of an elastic image in terms of whether or not the pressure operation on the object is appropriate using various kinds of data, which are used in the process of measuring the elastic information, and not displaying an elastic image, which does not need to be displayed, are known.

### CITATION LIST

### Patent Literature

[PTL 1] JP-A-2000-60853
[PTL 2] JP-A-2005-118152
[PTL 3] U.S. Pat. No. 6,558,324

### SUMMARY OF INVENTION

### Technical Problem

However, it is thought that improving the usability for an examiner when an elastic image is stored in a Cine memory or an external recording medium (hereinafter, these are appropriately referred to collectively as a memory) and is reproduced is not considered.

That is, the ultrasonic diagnostic apparatus can store various images including an elastic image, for example, in a Cine memory provided in the ultrasonic diagnostic apparatus or in an external storage medium, such as a DVD (Digital Versatile Disk), and reproduce and display these images later. The examiner performs diagnosis while reproducing various images stored in the Cine memory in the ultrasonic diagnostic apparatus or while reproducing various images stored in the external storage medium using a PC (Personal Computer), for example.

In this respect, in the above-described Patent Literatures, an elastic image which does not need to be displayed is not displayed when elastic images are displayed in real time. For this reason, storing an elastic image in a memory and reproducing the elastic image are not taken into consideration. Accordingly, even an elastic image which is not effective for diagnosis because the pressure state is not appropriate is stored in a memory and is reproduced. As a result, the efficiency of diagnosis performed by an examiner while observing reproduced elastic images becomes worse or the examiner needs to select an elastic image effective for diagnosis by performing frame feeding or skipping manually when elastic images which are not effective for diagnosis continue. This is not preferable when the usability for the examiner is taken into consideration.

Therefore, it is an object of the present invention to improve the usability for an examiner when an elastic image is stored in a memory and is reproduced.

### Solution to Problem

An ultrasonic diagnostic apparatus of the present invention which solves the above-described problems includes: an ultrasonic probe which transmits and receives an ultrasonic wave to and from an object; a phasing addition means that generates RF signal frame data of a sectional part of the object on the basis of a reflected echo signal measured by the ultrasonic probe; a displacement measurement means that generates displacement frame data by measuring displacement of tissue of the sectional part on the basis of a pair of RF signal frame data items acquired at different times; an elastic information calculation means that generates elastic frame data by calculating elastic information, which indicates hardness or softness of tissue of the sectional part, on the basis of the generated displacement frame data; an elastic image configuration means that generates an elastic image on the basis of the elastic frame data; an image display device which displays the elastic image; and a memory in which the elastic image is stored.

In particular, the ultrasonic diagnostic apparatus includes a pressure evaluating section which evaluates whether or not a pressure state of the object is appropriate on the basis of at least one of the displacement frame data of the tissue of the sectional part, the elastic frame data, and the pressure of the ultrasonic probe on an ultrasonic wave transmission/reception surface. The pressure evaluating section stores an elastic image in an appropriate pressure state in the memory selectively.

According to this, since elastic images generated in a state where the object is appropriately pressed are selectively stored in a memory, an elastic image suitable for diagnosis is selectively reproduced when an examiner performs diagnosis by reproducing the elastic image stored in the memory. Therefore, since time is not taken for frame feeding or skipping when elastic images which are not suitable for diagnosis continue, good usability for the examiner is realized and it is possible to shorten the diagnostic time by improving the diagnostic efficiency.

In addition, the pressure evaluating section may read and reproduce an elastic image in the appropriate pressure state selectively when reproducing elastic images stored in the memory. That is, even if all the elastic images are first stored in a memory and an elastic image in the appropriate pressure state is selectively read and reproduced when elastic images are reproduced instead of selectively storing an elastic image in the appropriate pressure state in the memory, good usability for the examiner is similarly realized. In addition, since the diagnostic efficiency is improved, it is possible to shorten the diagnostic time.

Here, whether or not the pressure state is appropriate is evaluated on the basis of points of view, such as whether or not the amount of pressure on the object or the pressure speed is appropriate, whether pressure is not performed in a state where the ultrasonic probe is inclined in a beam line direction with respect to the body surface of the object (in a biased pressure state), or whether the pressure operation itself is performed.

### Advantageous Effects of Invention

According to the present invention, it is possible to improve the usability for an examiner when an elastic image is stored in a memory and is reproduced.

### BRIEF DESCRIPTION OF DRAWINGS

FIG. 1 is a view showing the entire configuration of an ultrasonic diagnostic apparatus of the present embodiment.

FIG. 2 is a conceptual view showing a state of storage of an elastic image in a memory in a conventional ultrasonic diagnostic apparatus.

FIG. 3 is a view showing the concept of the processing content of a pressure evaluating section.

FIG. 4 is a view showing the relationship between the amount of pressure and the technique of an examiner when acquiring an elastic image.

FIG. 5 is a conceptual view showing the processing content of a second example of a pressure evaluating section.

FIG. 6 is a conceptual view showing the processing content of the second example of the pressure evaluating section.

FIG. 7 is a graph showing a temporal change in pressure data.

FIG. 8 is a conceptual view showing the processing content of a third example of the pressure evaluating section.

FIG. 9 is a conceptual view showing the processing content of the third example of the pressure evaluating section.

FIG. 10 is a conceptual view showing the processing content of a fourth example of the pressure evaluating section.

FIG. 11 is a conceptual view showing the processing content of the fourth example of the pressure evaluating section.

FIG. 12 is a conceptual view showing the processing content of a fifth example of the pressure evaluating section.

FIG. 13 is a conceptual view showing the processing content of the fifth example of the pressure evaluating section.

FIG. 14 is a conceptual view showing the processing content of the fifth example of the pressure evaluating section.

FIG. 15 is a conceptual view showing the processing content of a sixth example of the pressure evaluating section.

FIG. 16 is a conceptual view showing the processing content of the sixth example of the pressure evaluating section.

FIG. 17 is a conceptual view showing the processing content of a seventh example of the pressure evaluating section.

FIG. 18 is a conceptual view showing the processing content of an eighth example of the pressure evaluating section.

FIG. 19 is a conceptual view showing the processing content of the eighth example of the pressure evaluating section.

### DESCRIPTION OF EMBODIMENTS

Hereinafter, embodiments of an ultrasonic diagnostic apparatus, an elastic image storage/reproduction method, and an elastic image storage/reproduction program to which the present invention is applied will be described. In addition, in the following explanation, the same reference numerals are given to the same functional components, and repeated explanation thereof will be omitted.

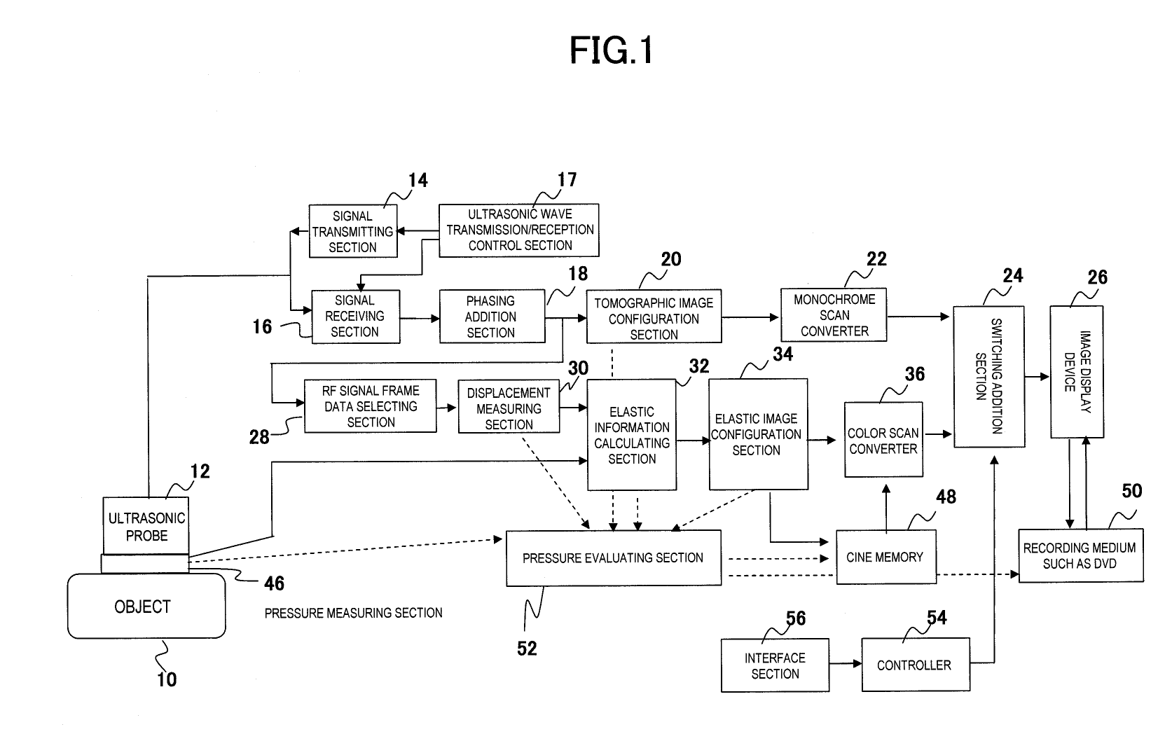

FIG. 1 is a view showing the entire configuration of an ultrasonic diagnostic apparatus of the present embodiment. As shown in FIG. 1, an ultrasonic diagnostic apparatus 100 includes: an ultrasonic probe 12 used in contact with an object 10; a signal transmitting section 14 which transmits an ultrasonic wave repeatedly to the object 10 through the ultrasonic probe 12 at intervals; a signal receiving section 16 which receives time-series reflected echo signals generated from the object 10; an ultrasonic wave transmission/reception control section 17 which controls the signal transmitting section 14 and the signal receiving section 16; a phasing addition section 18 which performs phasing addition of the received reflected echoes to generate RF signal frame data in time series; a tomographic image configuration section 20 which generates a gray-scale tomographic image, for example, a monochrome tomographic image by performing various kinds of signal processing on the RF signal frame data objected to the phasing addition by the phasing addition section 18; and a monochrome scan converter 22 which converts an output signal of the tomographic image configuration section 20 so as to fit the display of an image display device 26.

In addition, the ultrasonic diagnostic apparatus 100 includes: an RF signal frame data selecting section 28 which selects a pair of RF signal frame data items, which are measured at different times, from the RF signal frame data output from the phasing addition section 18; a displacement measuring section 30 which measures displacement caused in body tissue of the object 10 on the basis of the pair of RF signal frame data items and generates displacement frame data; an elastic information calculating section 32 which generates elastic frame data by calculating elastic information (distortion or an elastic modulus), which indicates the hardness or softness of body tissue of the object in a continuous pressure process, on the basis of the displacement frame data measured by the displacement measuring section 30; an elastic image configuration section 34 which generates an elastic image from the distortion or the elastic modulus calculated by the elastic information calculating section 32; and a color scan converter 36 which converts an output signal of the elastic image configuration section 34 so as to fit the display of the image display device 26.

In addition, the ultrasonic diagnostic apparatus 100 includes: a pressure measuring section 46, such as a pressure sensor, which measures the pressure of the ultrasonic probe 12 on the ultrasonic wave transmission/reception surface; a Cine memory 48 in which the elastic image generated by the elastic image configuration section 34 is stored and which is provided in the ultrasonic diagnostic apparatus; a recording medium 50, such as a DVD, in which the elastic image generated by the elastic image configuration section 34 is stored; and a pressure evaluating section 52 which evaluates whether or not the pressure state of the object 10 is appropriate on the basis of at least one of the displacement frame data generated by the displacement measuring section 30, the elastic frame data generated by the elastic information calculating section 32, and the pressure measured by the pressure measuring section 46 and which is a characteristic configuration of the present embodiment. Details of the pressure evaluating section 52 will be described later. In addition, in this specification, the Cine memory 48 and the recording medium 50, such as a VCR or a DVD, are collectively called a memory. Accordingly, memory simply referred to in this specification includes at least one the Cine memory 48 and the recording medium 50, such as a VCR or a DVD.

In addition, the ultrasonic diagnostic apparatus 100 includes a controller 54 which controls each of the constituent components described above, for example, a CPU (Central Processing Unit) and an interface section 56, such as a mouse, a keyboard, a touch panel, or a track ball, which gives an instruction to control a tint or an ROI (Region Of Interest) of an elastic image, a frame rate, and the like.

The ultrasonic probe 12 is formed by arraying plural transducers, and has a function of performing beam scanning electronically to transmit and receive an ultrasonic wave to and from the object 10 through the transducers. The signal transmitting section 14 has a function of generating a transmission wave pulse for generating an ultrasonic wave by driving the ultrasonic probe 12 and setting the convergent point of transmitted ultrasonic waves at a certain depth. In addition, the signal receiving section 16 generates an RF signal, that is, a reception wave signal by amplifying the reflected echo signal received by the ultrasonic probe 12 with a predetermined gain. The phasing addition section 18 controls the phase of the input RF signal amplified by the signal receiving section 16, and generates RF signal frame data by forming ultrasonic beams converged at plural convergent points.

The tomographic image configuration section 20 generates a gray-scale tomographic image of the object, for example, a monochrome tomographic image of the object on the basis of the RF signal frame data from the phasing addition section 18. The monochrome scan converter 22 acquires the tomographic frame data in the object 10, which is stored in a frame memory, as one image and reads the acquired tomographic frame data in synchronization with a television.

The monochrome scan converter 22 acquires RE signal frame data within the object 10 including moving tissue at periods of ultrasonic waves and converts the frame data into an image and displays it.

The switching addition section 24 is configured to include a frame memory, an image processing section, and an image selecting section. Here, the frame memory stores a tomographic image from the monochrome scan converter 22 and an elastic image from the color scan converter 36. In addition, the image processing section combines the tomographic image and the elastic image, which are secured in the frame memory, at the set ratio according to the command of the controller 54. The brightness information and the color information regarding each pixel of the composite image are acquired by adding the information on the monochrome tomographic image and the information on the color elastic image at the set ratio. In addition, the image selecting section selects an image, which is to be displayed on the image display device 26, from the tomographic image and the elastic image in the frame memory and the composite image of the image processing section according to the command of the controller 54. In addition, the tomographic image and the elastic image may be separately displayed without being combined.

The RF signal frame data selecting section 28 is configured to include a frame memory and a selection section. The RF signal frame data selecting section 28 stores plural RF signal frame data items from the phasing addition section 18 in a frame memory and selects a pair of RF signal frame data items, that is, two items of the RF signal frame data from the stored RF signal frame data group. For example, the RF signal frame data selecting section 28 secures RF signal data generated in time series from the phasing addition section 18, that is, generated on the basis of a frame rate of an image in a frame memory sequentially and selects the currently secured RF signal frame data (N) as first data according to the command from the controller 54 using the selection section and also selects one RF signal frame data item (X) from the RF signal frame data group (N-1, N-2, N-3, N-M) stored in the past. In addition, N, M, and X herein are index numbers given to the RF signal frame data, and are assumed to be natural numbers.

The displacement measuring section 30 calculates the displacement of body tissue and the like from the pair of RF signal frame data items. For example, the displacement measuring section 30 performs one-dimensional or two-dimensional correlation processing on the pair of data items selected by the RF signal frame data selecting section 28, that is, the RF signal frame data (N) and the RF signal frame data (X) to calculate a displacement or movement vector in body tissue corresponding to each point of the tomographic image, that is, one-dimensional or two-dimensional displacement distribution regarding the displacement direction and size. Here, in order to detect the movement vector, a block matching method is used. The block matching method is to perform processing in which an image is divided into blocks with, for example, “N×N” pixels, a block in a region of interest is observed, the most similar block to the observed block is searched for from previous frames, and a sample value is determined by predictive coding, that is, by the difference referring to this.

The elastic information calculating section 32 generates elastic frame data by calculating elastic information (distortion or an elastic modulus), which indicates the hardness or softness of body tissue of the object in a continuous pressure process, on the basis of the displacement frame data measured by the displacement measuring section 30. In this case, distortion data is calculated by spatial differentiation of the amount of movement of body tissue, for example, by spatial differentiation of the displacement. In addition, data of the elastic modulus is calculated by dividing the pressure change by the change in the amount of movement. For example, assuming that the displacement measured by the displacement measuring section 30 is ΔL and the pressure measured by the pressure measuring section 46 is ΔP, distortion (S) can be calculated by spatial differentiation of ΔL. Accordingly, the distortion (S) can be calculated using Expression S=ΔL/ΔX. In addition, Young's modulus m of elastic modulus data is calculated by Expression of m=(ΔP)/(ΔL/L). Since the elastic modulus of body tissue corresponding to each point of a tomographic image is calculated from this Young's modulus m, it is possible to acquire the two-dimensional elastic image data continuously. In addition, Young's modulus is a ratio of simple tensile stress applied to the body to the tensile strain occurring in parallel to the tensile stress.

The elastic image configuration section 34 is configured to include a frame memory and an image processing section. The elastic image configuration section 34 secures elastic frame data, which is output in time series from the elastic information calculating section 32, in a frame memory and performs image processing on the secured elastic frame data using the image processing section.

The color scan converter 36 converts the frame data into color information on the basis of the elastic image data from the elastic image configuration section 34. That is, the color scan converter 36 converts the frame data into three primary colors of light, that is, red (R), green (G), and blue (B) on the basis of the elastic image data. For example, the color scan converter 36 converts the data of an elastic image with large distortion into a red code and also converts the data of an elastic image with small distortion into a blue code. In addition, the gradation of red (R), green (G), and blue (B) is 256 levels, and 255 means displaying with a maximum brightness and 0 means being not displayed on the contrary.

The operation of the ultrasonic diagnostic apparatus 100 configured in this way will be described. In the ultrasonic diagnostic apparatus 100, the signal transmitting section 14 transmits an ultrasonic wave repeatedly to the object 10 at intervals through the ultrasonic probe 12 being in contact with the object 10, the signal receiving section 16 receives time-series reflected echo signals generated from the object 10, and phasing addition is performed on the reflected echo signals to generate RF signal frame data. On the basis of the RF signal frame data, a gray-scale tomographic image, for example, a monochrome B-mode image is acquired by the tomographic image configuration section 20. In this case, when the ultrasonic probe 12 is scanned in a fixed direction, one tomographic image is acquired. On the other hand, an elastic image is acquired by the elastic image configuration section 34 on the basis of the RF signal frame data after performing phasing addition by the phasing addition section 18. Then, a composite image is generated by adding the acquired monochrome tomographic image and the acquired color elastic image by the switching addition section 24.

Meanwhile, in the ultrasonic diagnostic apparatus 100 capable of generating such an elastic image, it is possible to store a tomographic image and an elastic image automatically in the Cine memory 48 in time series or to store a tomographic image and an elastic image in the recording medium 50, such as a DVD, in time series. In addition, it is possible to perform diagnosis while reproducing the tomographic image and the elastic image, which are stored in the Cine memory 48, in the ultrasonic diagnostic apparatus or while reproducing the tomographic image and the elastic image, which are stored in the recording medium 50, using a PC, for example.

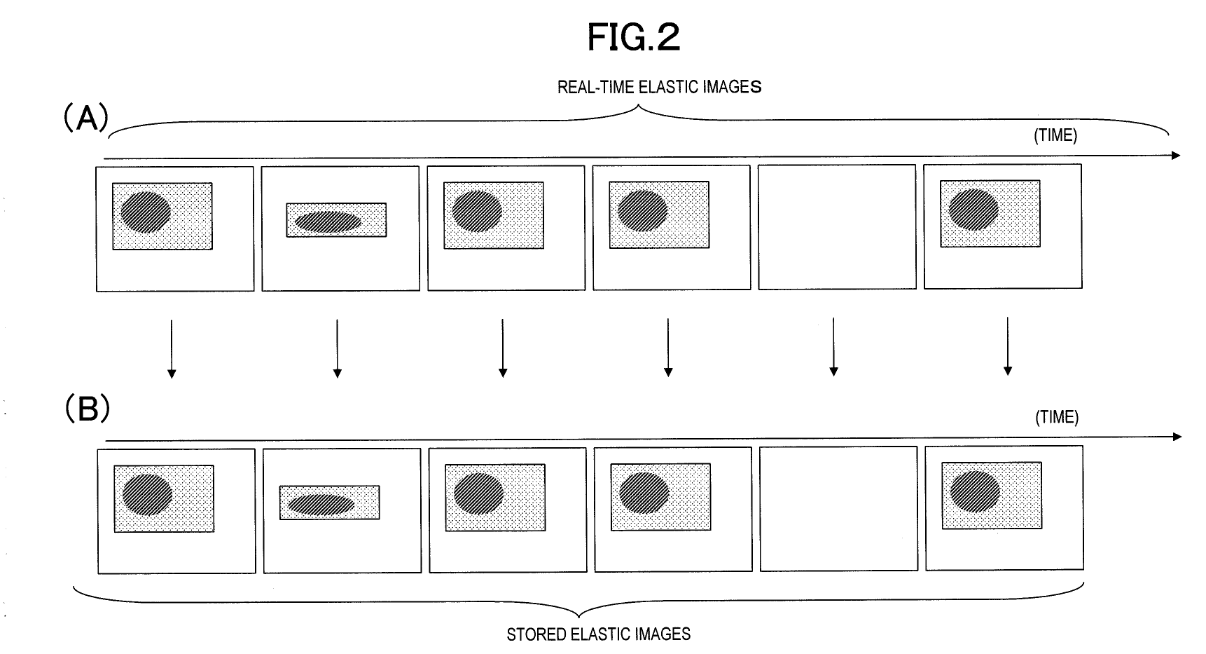

Here, FIG. 2 is a conceptual view showing a state of storage of an elastic image in a memory in a conventional ultrasonic diagnostic apparatus. FIG. 2(A) shows the concept of a real-time elastic image generated in the ultrasonic diagnostic apparatus 100, and FIG. 2(B) is a view showing the concept of an elastic image stored in a memory. As shown in FIG. 2(A), it is assumed that elastic images which are not effective for diagnosis are generated, for example, in second and fifth frames because a pressure state is not appropriate.

That is, in order to acquire an elastic image which is effective for diagnosis, it is necessary to cause tissue of a sectional part to be properly displaced by pressing the object appropriately on the ultrasonic wave transmission/reception surface of the ultrasonic probe 12, for example. However, an elastic image which is not effective for diagnosis may be generated because pressure is performed in a state where the ultrasonic probe 12 is inclined in a beam line direction with respect to the body surface of the object 10 (in a biased pressure state), in which the amount of the pressure onto the object 10 and the pressure speed are not appropriate, or because the pressure operation itself is not performed, for example. In a conventional ultrasonic diagnostic apparatus, since all generated elastic images are stored in a memory as shown in FIG. 2(B), an elastic image which is not effective for diagnosis is also included in the elastic images.

As a result, when the efficiency of diagnosis performed by an examiner while observing reproduced elastic images becomes worse or when elastic images which are not effective for diagnosis continue, the examiner needs to select an elastic image effective for diagnosis by performing frame feeding or skipping manually. This is not preferable when the usability for the examiner is taken into consideration.

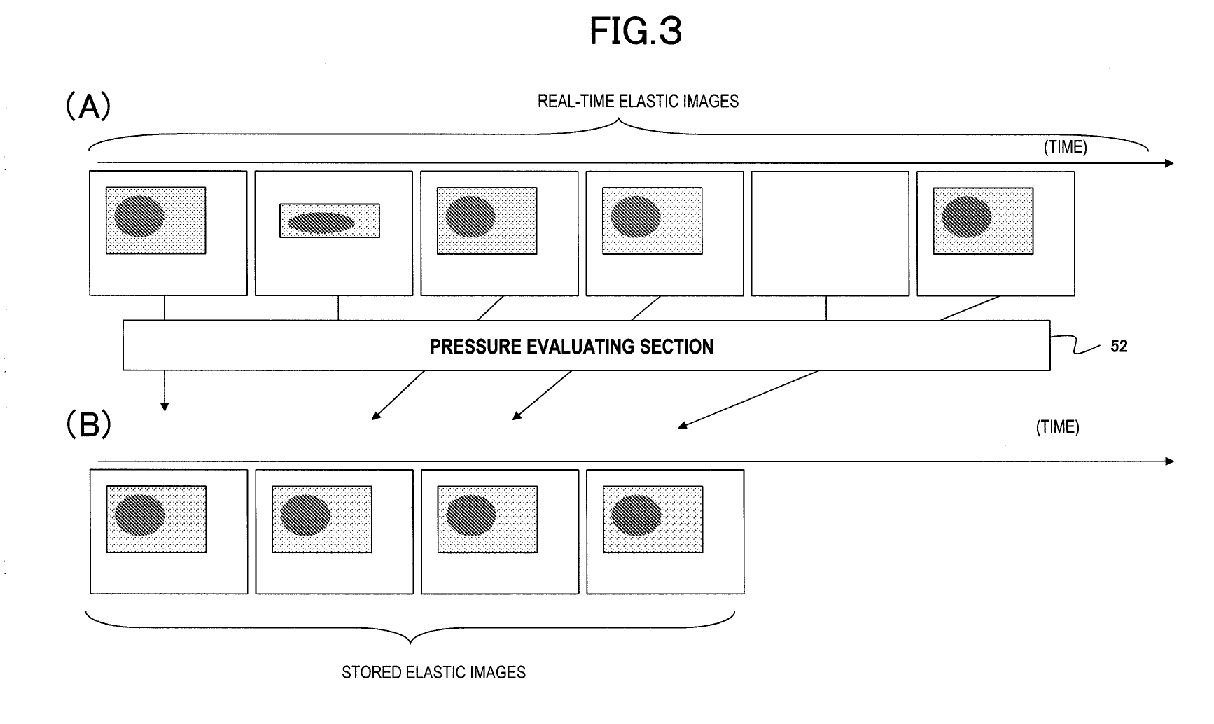

Therefore, the pressure evaluating section 52 is provided in the ultrasonic diagnostic apparatus of the present embodiment. FIG. 3 is a view showing the concept of the processing content of the pressure evaluating section 52. FIG. 3(A) shows the concept of a real-time elastic image generated in the ultrasonic diagnostic apparatus 100, and FIG. 3(B) is a view showing the concept of an elastic image stored in a memory. Similar to the case shown in FIG. 2, it is assumed that elastic images, which are not effective for diagnosis, are generated, for example, in second and fifth frames because a pressure state is not appropriate.

First, the pressure evaluating section 52 evaluates whether or not the pressure state of the object 10 is appropriate on the basis of at least one of the displacement frame data and the elastic frame data of the tissue of the sectional part of the object 10 and the pressure measured by the pressure measuring section 46. A specific evaluation method will be described in detail later. Then, as shown in FIG. 3(B), elastic images in an appropriate pressure state, that is, elastic images of first, third, fourth, and sixth frames in FIG. 3(A) are selectively stored as elastic images of the first to fourth frames in a memory.

According to this, since elastic images generated in a state where the object 10 is appropriately pressed are selectively stored in a memory, an elastic image suitable for diagnosis is selectively reproduced when an examiner performs diagnosis by reproducing the elastic image stored in the memory. Therefore, since it does not take time for frame feeding or skipping when elastic images which are not suitable for diagnosis continue, good usability for the examiner is realized and it is possible to shorten the diagnostic time by improving the diagnostic efficiency.

Hereinafter, a specific evaluation method of the pressure evaluating section 52 will be described through each example. In addition, in the following examples, a case where an elastic image in an appropriate pressure state is selectively stored in a memory will be described. However, when reproducing elastic images stored in a memory, an elastic image in an appropriate pressure state may also be selectively read and reproduced. Similarly, in this case, good usability for the examiner is realized and it is possible to shorten the diagnostic time by improving the diagnostic efficiency.

In addition, when the pressure evaluating section 52 stores an elastic image in an appropriate pressure state in a memory selectively or reads and reproduces the elastic image in the appropriate pressure state from the memory selectively, a tomographic image of a frame corresponding to the elastic image in the appropriate pressure state may be similarly stored in the memory or may be similarly read and reproduced from the memory for the tomographic image. In this specification, the “pressure evaluating section 52 stores an elastic image in an appropriate pressure state in a memory selectively or reads and reproduces the elastic image in the appropriate pressure state from the memory selectively” is assumed to include storing not only the elastic image in the appropriate pressure state but also a tomographic image of a frame, which corresponds to the elastic image in the appropriate pressure state, in the memory or reading and reproducing not only the elastic image in the appropriate pressure state but also the tomographic image of the frame from the memory. In the following explanation, a case where a tomographic image and an elastic image in an appropriate pressure state are selectively stored in a memory will be described as an example.

### First Example

In a first example, it is evaluated that the pressure state is appropriate when at least one of the variance or deviation of displacement of plural measurement points of displacement frame data and the variance or deviation of elastic information of plural measurement points of elastic frame data is smaller than the threshold value set in advance. That is, a fine pressure operation is required in order to acquire an elastic image which is generally effective for diagnosis, and an elastic image which is not suitable for diagnosis is generated if excessive pressure is performed. In this example, therefore, in order to estimate an inappropriate elastic image from a variation in displacement, it is determined whether or not the pressure state is appropriate using statistical features, such as displacement in each frame, distortion, or an elastic modulus, and a tomographic image and an elastic image in the appropriate pressure state are selectively stored and reproduced.

For example, the amount of displacement in a pixel (i, j) (0≦i≦M, 0≦j≦N) of certain displacement frame data output by the displacement measuring section 30 is assumed to be l(i, j). Then, the variance uk of displacement of displacement frame data is expressed as the following Expression 1.

$$uk = \frac{1}{M * N} {\sum }_{i = 0} {\sum }_{j = 0} {( l ( i , j ) ave - l ( i , j ) )}^{2} \qquad [ Expression 1 ]$$

l(i, j)ave is an average of l(i, j) over the entire frame region. When fine pressure is repeated, a displacement change becomes small, and the variance is decreased accordingly. On the contrary, when excessive pressure is performed, the displacement change becomes large, and the variance is increased accordingly. Then, assuming that the threshold value set in advance is uth, the pressure evaluating section 52 stores a tomographic image and an elastic image in the Cine memory 48 or the recording medium 50, such as a DVD, only when uk<uth. The examiner can set an optimal value of Uth through the interface section 56 according to a diagnostic part, for example.

Although the variance is calculated over the entire frame in the above, it is also possible to calculate the variance of displacement of eight points adjacent to the pixel (i, j) as uk(i, j) and to store an elastic image only when ukave, which is an average of uk(i, j) over the entire frame region, becomes smaller than uth, for example. In addition, a tomographic image and an elastic image may also be stored only when the number of pixels satisfying uk(i, j)<uth becomes equal to or larger than a certain rate.

In addition, although the displacement is used as a reference parameter of pressure evaluation in the above, distortion or an elastic modulus of elastic frame data output from the elastic information calculating section 32 maybe used as a reference parameter, and a combination thereof may be used. This is because the distortion or the elastic modulus is calculated using displacement and the elastic frame data reflects a local discreteness of displacement frame data. In addition, it is possible to use not only the variance but also an average value, a deviation, or the like as statistical features.

### Second Example

Next, a second example will be described. A pressure evaluating section in this example evaluates that the pressure state is appropriate when an absolute value of at least one of the average value of displacement of plural measurement points of displacement frame data and the average value of elastic information of plural measurement points of elastic frame data is larger than the threshold value set in advance.

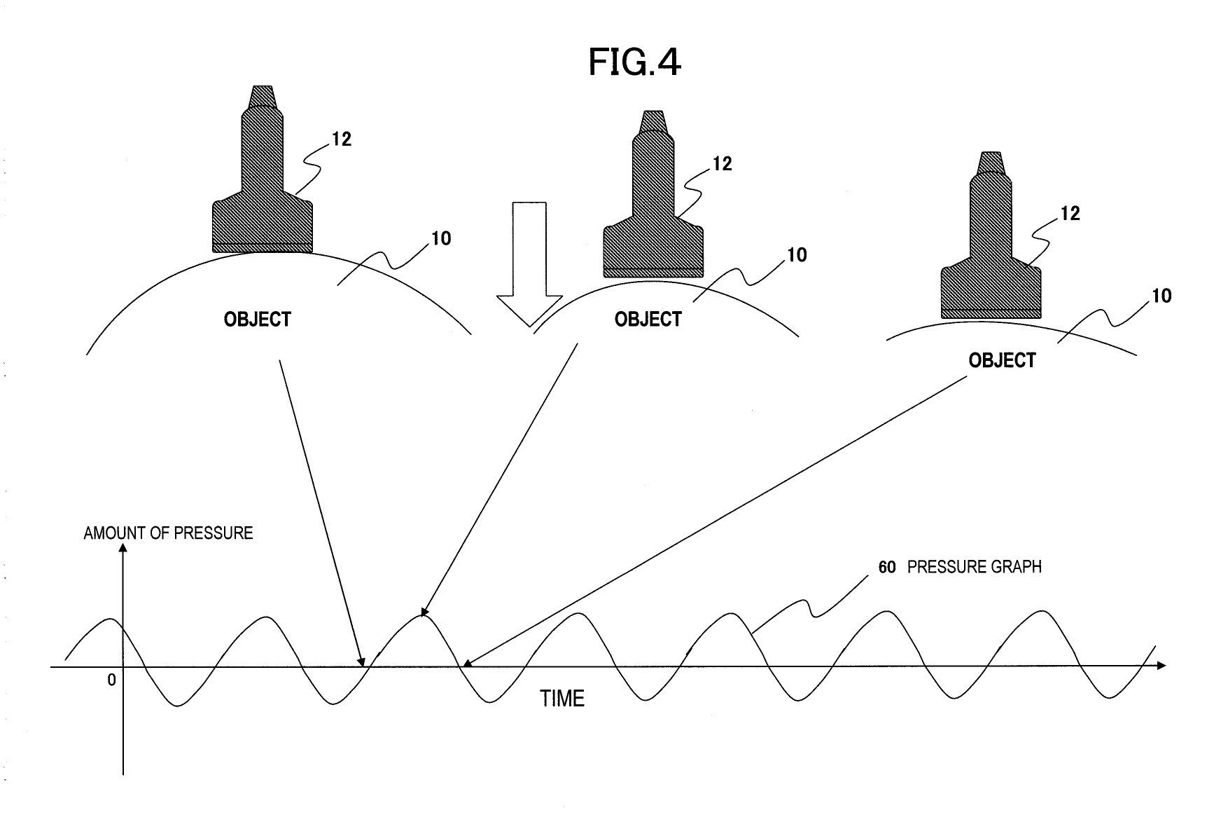

That is, FIG. 4 is a view showing the relationship between the amount of pressure and the technique of an examiner when acquiring an elastic image. As shown in FIG. 4, when capturing an elastic image using an ultrasonic diagnostic apparatus, the examiner presses the object 10 using the ultrasonic probe 12, and the elastic image can be acquired by repeating the pressure. For this reason, assuming that the average of displacement in each frame acquired by the displacement measuring section 30 or the average of distortion in each frame acquired by the elastic information calculating section 32 is the amount of pressure, a temporal change in the amount of pressure becomes a graph (hereinafter, referred to as a pressure graph 60) in FIG. 4. As shown in FIG. 4, when pressing the object 10 with the ultrasonic probe 12 or pulling the ultrasonic probe 12 from the object 10, that is, when the pressure speed is high, displacement or distortion becomes large in positive and negative directions (absolute value of the amount of pressure becomes large). On the contrary, when the ultrasonic probe 12 stops, that is, when the pressure speed becomes 0, the amount of pressure also becomes 0.

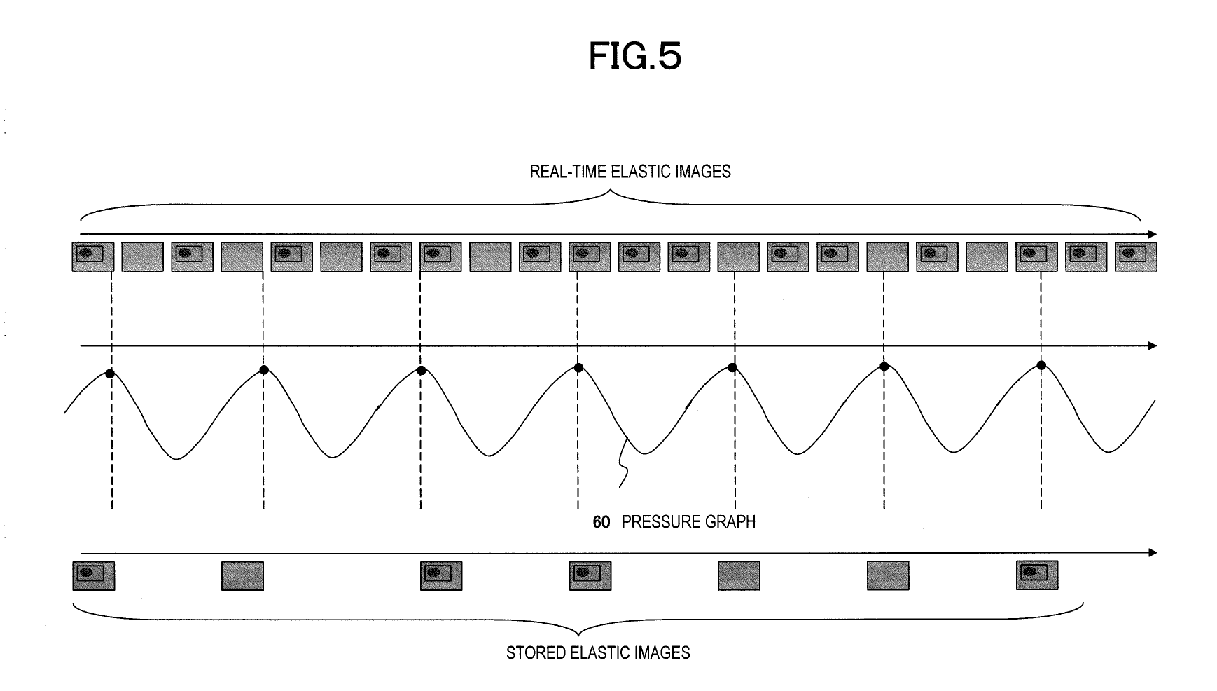

Therefore, in this example, the pressure state is evaluated using a method described in FIG. 5. FIG. 5 is a conceptual view showing the processing content of the pressure evaluating section 52. Since an elastic image is generated by displacement or distortion, it is possible to measure the size of displacement or distortion as the amount of pressure and evaluate whether or not the pressure state is appropriate on the basis of the amount of pressure. Therefore, as an example when the absolute value of the amount of pressure is larger than the threshold value set in advance as shown in FIG. 5, a time taking the maximum value or the minimum value of a pressure graph is detected by the pressure evaluating section 52, and a tomographic image and an elastic image at the time are selectively stored in the Cine memory 48 or the recording medium 50, such as a DVD.

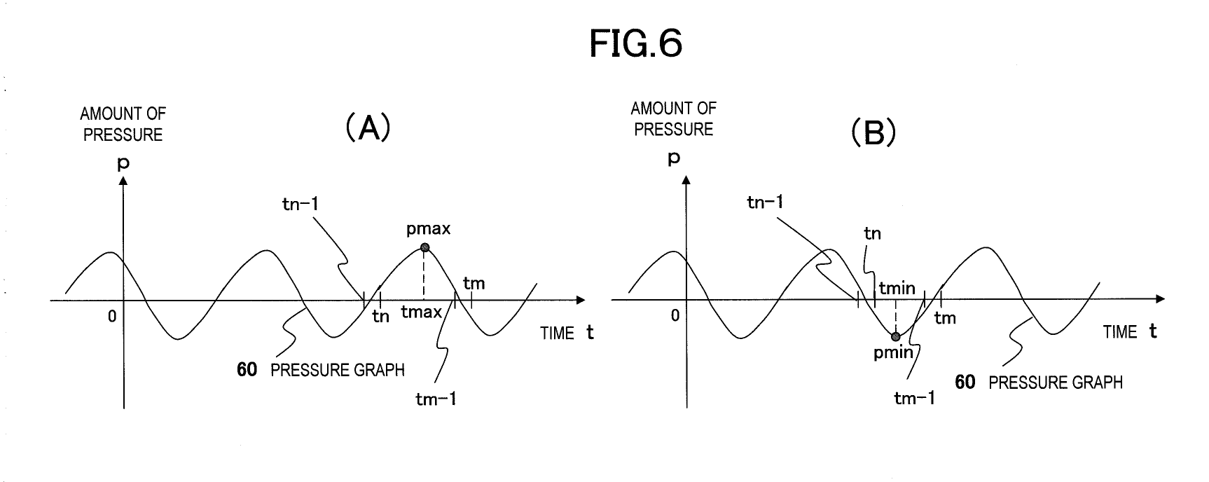

FIG. 6 is a view showing an example of the processing content of the pressure evaluating section 52. For example, as shown in FIGS. 6(A) and 6(B), it is assumed that the amount of pressure pn is acquired at a certain time tn. If pn×p(n−1)≦0, a point crossing 0 is shown in the pressure graph. Between tn at which pn×p(n−1)≦0 is satisfied and next tm at which pm×p(m−1)≦0 (m>n) is satisfied, the amount of pressure having a maximum value and a value of 0 or more is set as pmax and the amount of pressure having a minimum value and a value of 0 or less is set as pmin. Time at the time of pmax is set as tmax, and time at the time of pmin is set as tmin. The pressure evaluating section 52 repeats the above-described calculation in time series and stores a tomographic image and an elastic image at time tmax and tmin selectively in the Cine memory 48 or the recording medium 50, such as a DVD.

In addition, whether to store in the Cine memory 48 an image at tmin or tmax may be arbitrarily selected by external control of an examiner using an input interface, such as a keyboard. Thus, a tomographic image and an elastic image when the amount of pressure becomes a maximum value or a minimum value during the periodic temporal change in the amount of pressure can be selectively stored in the Cine memory 48 or the recording medium 50, such as a DVD. Alternatively, a tomographic image and an elastic image when the absolute value of the amount of pressure is larger than the threshold value set in advance can also be recorded selectively instead of selectively recording a tomographic image and an elastic image at tmax and tmin.

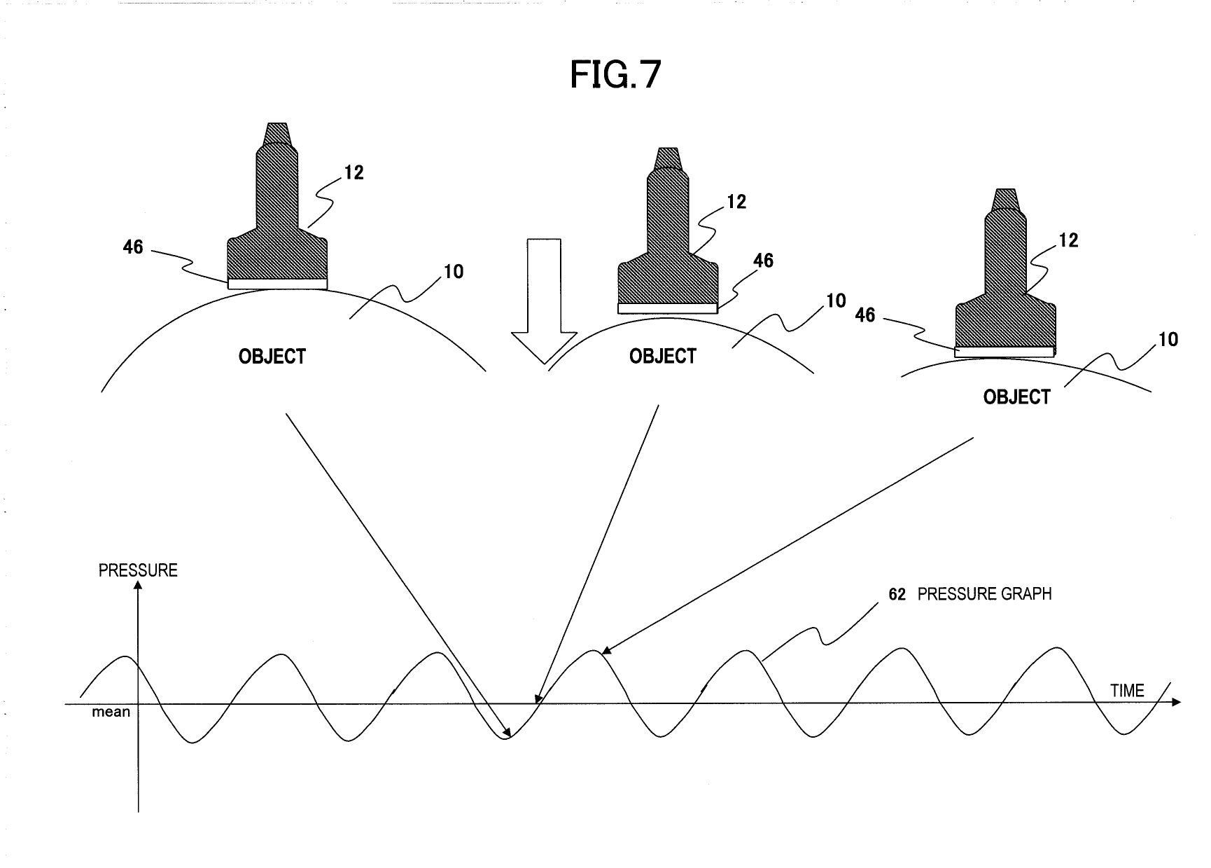

In addition, although the evaluation is performed on the basis of the amount of pressure in the above explanation, a temporal change in pressure data acquired by the pressure measuring section 46 may also be used. That is, it is possible to evaluate that the pressure state is appropriate when the absolute value of the rate of change of the pressure acquired by the pressure measuring section 46 is larger than the threshold value set in advance. FIG. 7 is a graph (hereinafter, referred to as a pressure graph 62) showing a temporal change in pressure data. Assuming that the average value of pressure data is a reference value, the pressure graph 62 crosses an average line when pressing the object with the ultrasonic probe 12, that is, when the pressure speed is high, as shown in the drawing. Therefore, only when the pressure during a periodic temporal change becomes equal to the average of pressure, a tomographic image and an elastic image may also be selectively stored in the Cine memory 48 or the recording medium 50, such as a DVD. Alternatively, a tomographic image and an elastic image when the pressure is in a range narrowed by the threshold value set in advance in the vertical direction with the average of pressure as a reference may also be selectively stored in the Cine memory 48 or the recording medium 50, such as a DVD.

### Third Example

Next, a third example of the present invention will be described. In this example, it is evaluated that the pressure state is appropriate when the variance or deviation of the pressure at plural places including at least both ends of the ultrasonic wave transmission/reception surface of the ultrasonic probe 12 in the beam line direction is smaller than the threshold value set in advance.

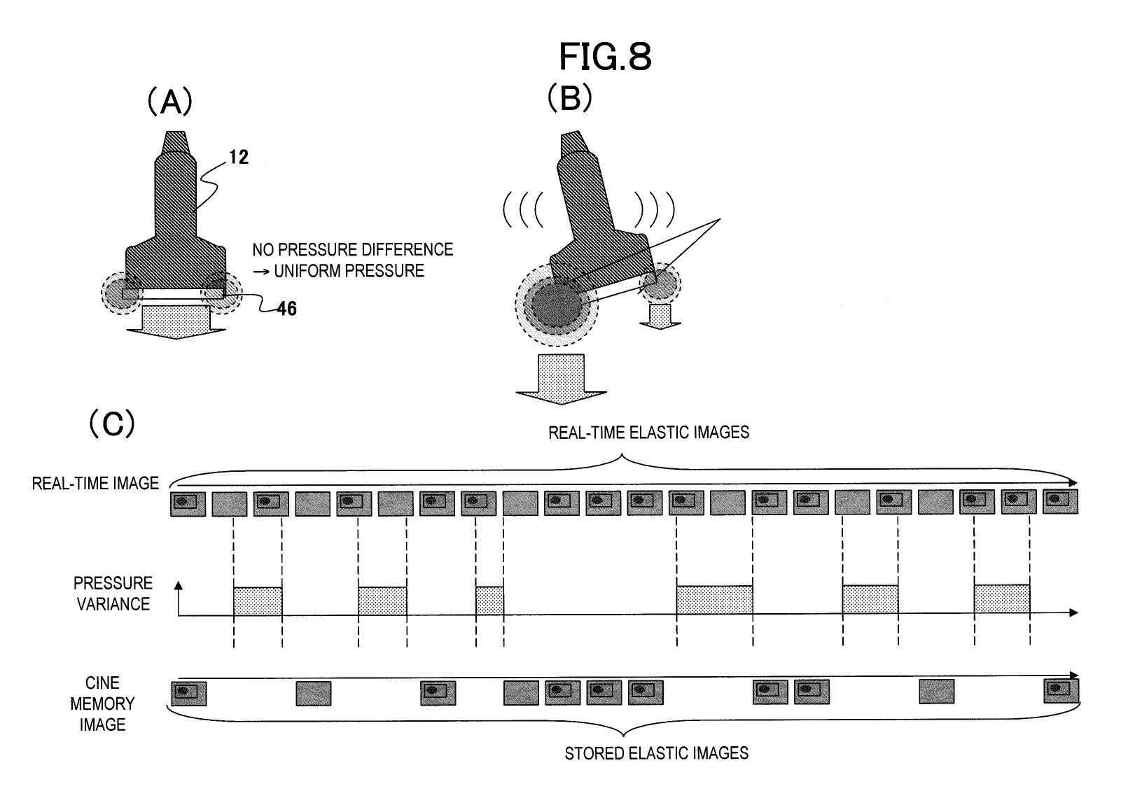

That is, in order to acquire an elastic image as described above, it is necessary to press the object 10 with the ultrasonic probe 12. However, a useful elastic image is not acquired if uniform pressure is not performed. FIG. 8(A) is a schematic view showing a state where uniform pressure is performed by the ultrasonic probe 12. In this case, a pressure difference between both ends of the ultrasonic wave transmission/reception surface of the ultrasonic probe 12 in the beam line direction becomes small.

On the other hand, FIG. 8(B) is a schematic view showing a state where pressure is performed in a state where the ultrasonic probe 12 is inclined in the beam line direction (horizontally inclined state). In this case, since the pressure difference between both ends of the ultrasonic wave transmission/reception surface of the ultrasonic probe 12 in the beam line direction becomes large, the horizontally inclined state can be detected. Accordingly, as shown in FIG. 8(C), the pressure evaluating section detects uniform pressure according to the pressure distribution between the ultrasonic probe 12 and the object 10 and stores corresponding images in the Cine memory 48 or the recording medium 50, such as a DVD, only at the time of uniform pressure.

More specifically, pressure measured by several pressure sensors provided on the surface of the ultrasonic probe 12 in contact with an object can be calculated by the pressure measuring section 46. Pressure acquired in each place is set as p(x, y) (0≦x≦X, 0≦y≦Y). x and y indicate the coordinates of the contact surface between the ultrasonic probe 12 and the object 10. Then, the average value pm and the variance pv (pressure variance) of pressure on the contact surface are acquired by the following Expressions 2 and 3, respectively.

$$\begin{matrix}pm = \frac{1}{x * y} {\sum }_{i = 0}^{X} {\sum }_{j = 0}^{Y} p ( i , j ) & [ Expression 2 ] \\ pv = \frac{1}{x * y} {\sum }_{i = 0}^{X} {\sum }_{j = 0}^{Y} {( pv - p ( i , j ) )}^{2} & [ Expression 3 ]\end{matrix}$$

In addition, the standard deviation pa is acquired by the following Expression 4.

pσ=\sqrt{pv} [Expression 4]

It is thought that the size of pressure changes with a place when the variance and the deviation are large. On the other hand, it is thought that a variation in the size of pressure according to a place is small when the variance and the deviation are small. Accordingly, assuming that the variance as a threshold value set in advance is vth and the standard deviation is ath, the pressure evaluating section 52 stores a tomographic image and an elastic image at the time of pv<vth or pσ<σth selectively in the Cine memory 48 or the recording medium 50, such as a DVD. In addition, it is also possible to use values other than the variance and the deviation as long as they are statistic values through which the pressure distribution of the ultrasonic probe contact surface can be quantitatively evaluated.

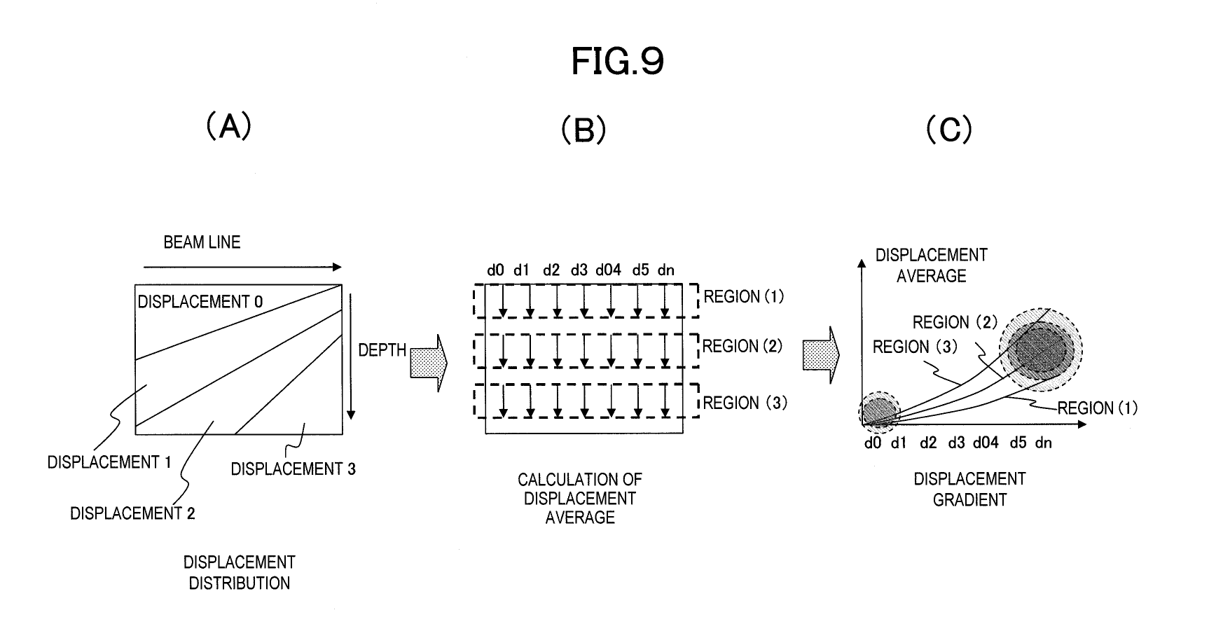

In addition, in the above explanation, a pressure variation is detected by the pressure acquired from the pressure measuring section 46. However, it is also possible to use elements other than the pressure as long as they are elements through which being pressed in a state where the ultrasonic probe 12 is inclined can be detected. For example, when the object 10 is pressed in a state where the ultrasonic probe 12 is inclined, displacement on the object 10 becomes non-uniform as shown in FIG. 9(A). For this reason, in three regions (1) to (3) divided in the depth direction on the object 10, displacement acquired from the displacement measuring section 30 for each of beam lines dO to do is equalized as shown in FIG. 9(B), for example. In addition, it can be determined that pressure is appropriate when the size of a gradient of the displacement average is smaller than the threshold value set in advance, as shown in FIG. 9(C). It is also possible to adopt a variance or deviation of the displacement average in the beam line direction instead of the size of the gradient of the displacement average and to store and reproduce an elastic image only when the variance or the deviation becomes smaller than a predetermined value.

### Fourth example

Next, a fourth example will be described. In this example, a correlation between at least one of a graph showing a temporal change in the pressure of the ultrasonic probe 12 on the ultrasonic wave transmission/reception surface, a graph showing a temporal change in the displacement of tissue of a sectional part, and a graph showing a temporal change in the elastic information of tissue of a sectional part and a corresponding one of an optimal pressure graph, an optimal displacement graph, and an optimal elastic information graph, which are set in advance for the graphs, is calculated, and it is evaluated that the pressure state is appropriate when the correlation is larger than the threshold value set in advance.

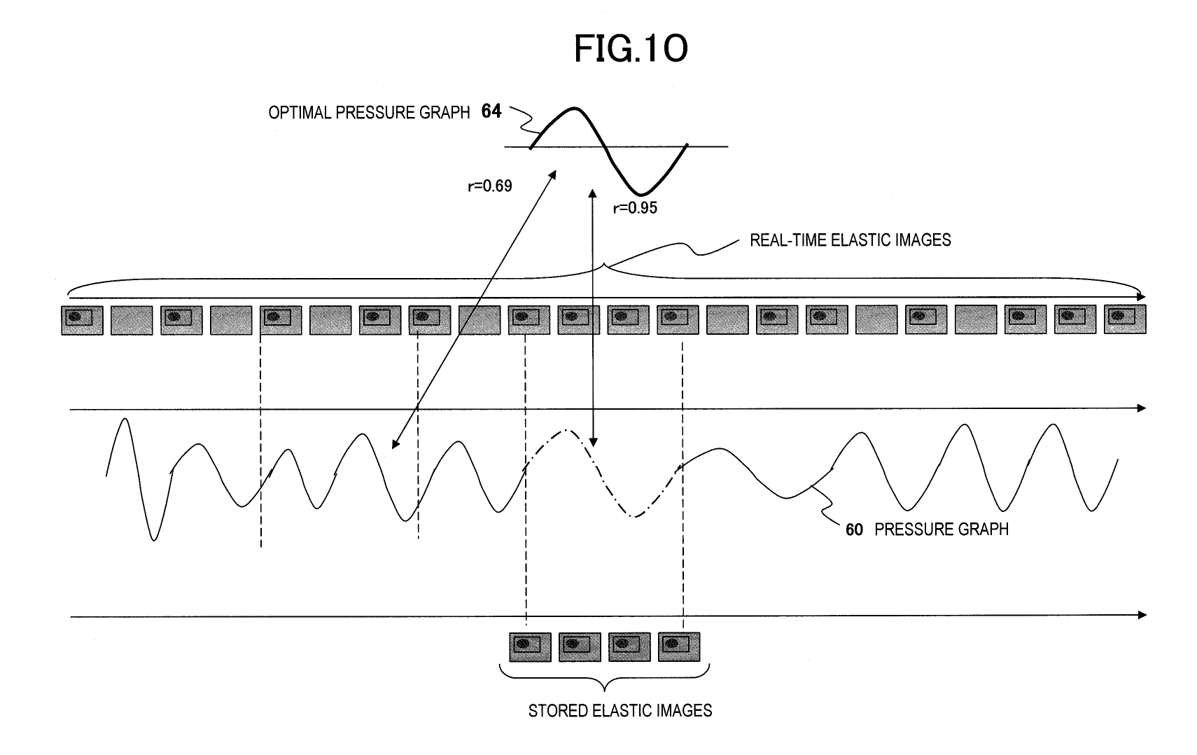

That is, since it is necessary to perform optimal pressure repeatedly in order to acquire a useful elastic image, the pressure graph 60 in FIG. 4 can be the guide. Therefore, a correlation between a pressure graph when performing an optimal pressure operation for diagnosis which is prepared in advance as shown in FIG. 10 (hereinafter, referred to as an optimal pressure graph 64) and the pressure graph 60 in the actual operation is taken, and only a portion with a large correlation is stored in the Cine memory 48 or the like. By this method, an elastic image close to the optimal pressure operation can be stored selectively in the Cine memory 48 or the recording medium 50, such as a DVD.

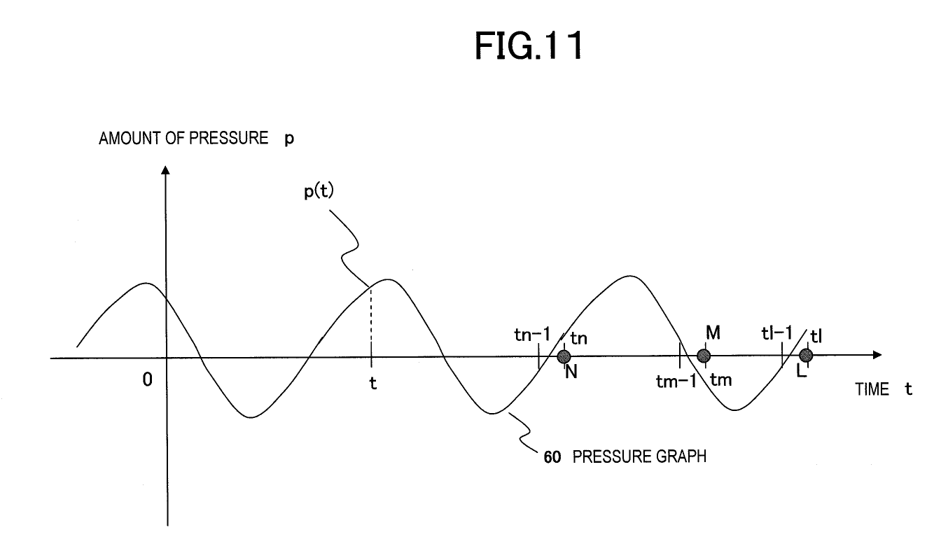

More specifically, p(tn)×p(tn−1)≦0 is satisfied at a point of the pressure graph crossing p=0 as shown in FIG. 11, as described above. It is assumed that this point is an N point, a crossing point after the N point is M, and a crossing point after the M point is an L point. In this case, a time between N and L becomes one period of the pressure graph. Then, data of the amount of pressure from N to L is set as population P(t), and data of the amount of pressure in one period of the optimal pressure graph set in advance is set as population Po(t).

Assuming that a correlation between P(t) and P₀(t) is Co, Co can be calculated by the following Expression 5.

$${C}_{0} = \frac{\frac{{\sum }_{i = 0}^{L - N} ( P ( i ) - Pave ) ( {P}_{0} ( i ) - {P}_{0} ave )}{L - N}}{\sqrt{\frac{{\sum }_{i = 0}^{L - N} {( P ( i ) - Pave )}^{2}}{L - N}} \sqrt{\frac{{\sum }_{i = 0}^{L - N} {( {P}_{0} ( i ) - {P}_{0} ave )}^{2}}{L - N}}} \qquad [ Expression 5 ]$$

In addition, Pave and POave are averages of P (t) and P₀(t) in a section from L to N, respectively. Only when Co is higher than a reference value Cstd set in advance, the pressure evaluating section 52 stores a tomographic image and an elastic image in the Cine memory 48 or the recording medium 50, such as a DVD. In addition, it is also possible to calculate a correlation between the pressure graph 62 showing a temporal change in pressure of the ultrasonic probe 12 on the ultrasonic wave transmission/reception surface and the optimal pressure graph set in advance and to store a tomographic image and an elastic image in the Cine memory 48 or the recording medium 50, such as a DVD, only when the correlation is larger than the threshold value set in advance.

### Fifth Example

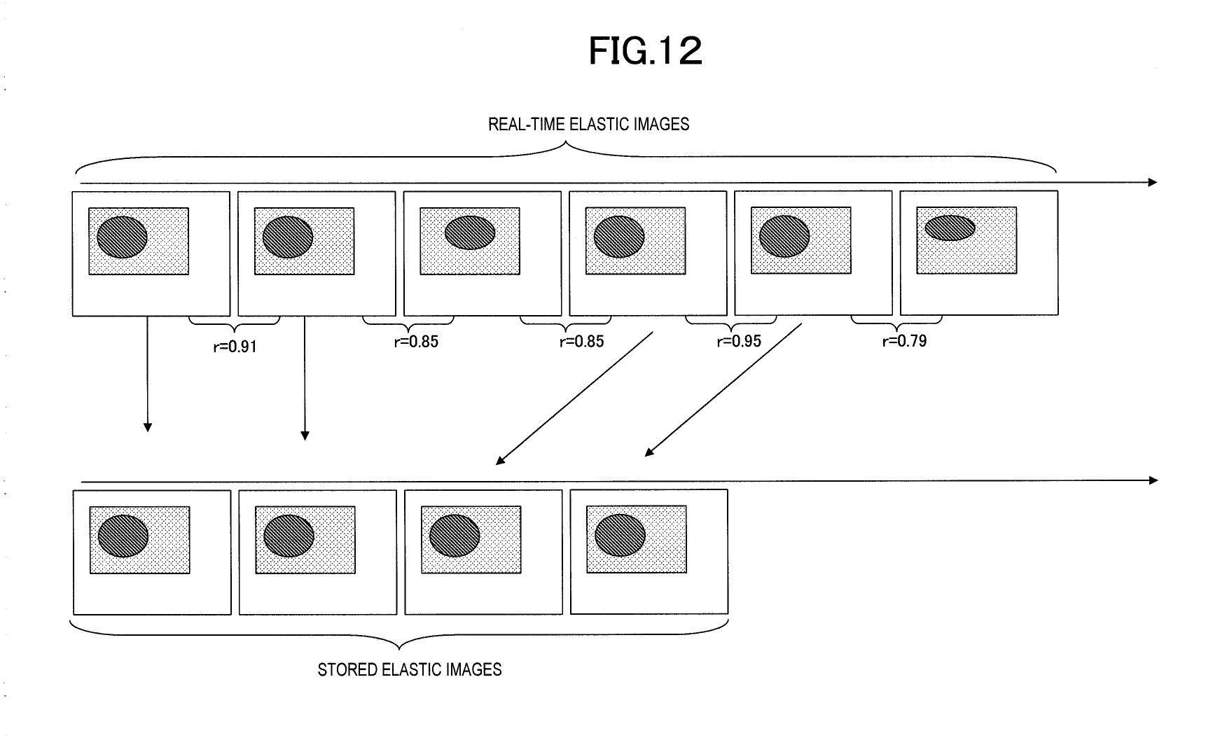

Next, a fifth example of the present invention will be described. In this example, a correlation between a pair of frames, which are adjacent to each other in time series, of either displacement frame data or elastic frame data is calculated and it is evaluated that the pressure state is appropriate when the correlation is larger than the threshold value set in advance. That is, a correlation between elements of a pair of elastic frame data items adjacent to each other in time series is calculated and a pair of a tomographic image and an elastic image, which are highly correlated, are stored in the Cine memory 48 or the recording medium 50, such as a DVD, as shown in FIG. 12, for example.

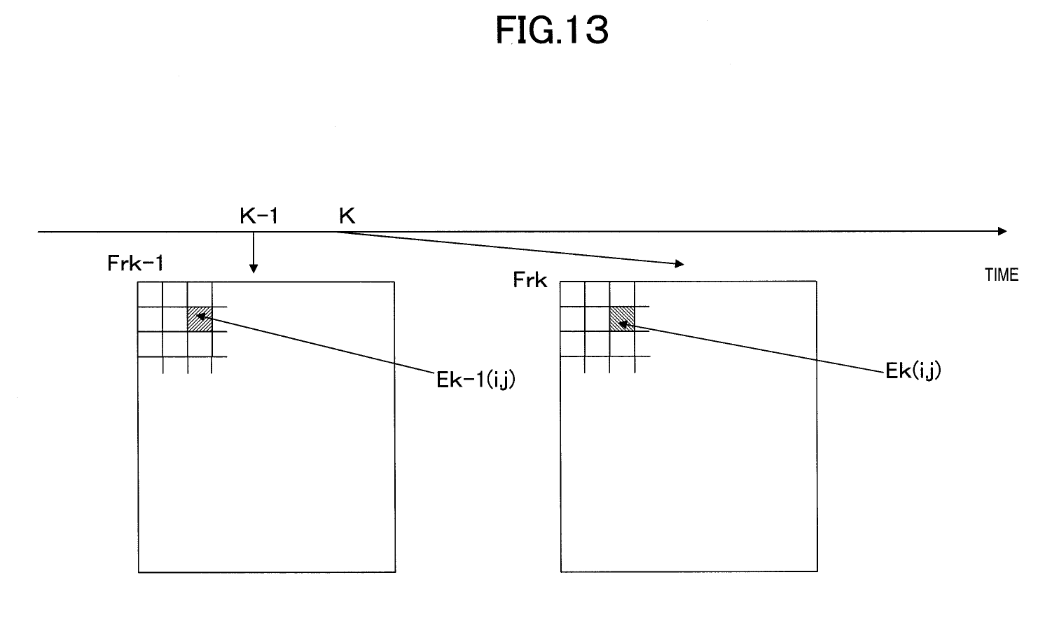

More specifically, elastic frame data output at time tk, among the elastic frame data output from the elastic information calculating section 32, is set as Frk and each element of the elastic frame data is set as Ek(i, j) (0≦i≦N, 0≦j≦M), as shown in FIG. 13. The pressure evaluating section 52 calculates a correlation Ck between Ek(i, j) and Ek−1(i, j) in all items of element data using the following Expression 6.

$$Ck = \frac{\frac{\begin{matrix}{\sum }_{i = 0}^{N} {\sum }_{j = 0}^{M} ( {E}_{k} ( i , j ) - {E}_{k} ave ) \\ ( {E}_{k - 1} ( i , j ) - {E}_{k - 1} ave )\end{matrix}}{N * M}}{\begin{matrix}\sqrt{\frac{{\sum }_{i = 0}^{N} {\sum }_{j = 0}^{M} {( {E}_{k} ( i , j ) - {E}_{k} ave )}^{2}}{N * M}} \\ \sqrt{\frac{{\sum }_{i = 0}^{N} {\sum }_{j = 0}^{M} {( {E}_{k - 1} ( i , j ) - {E}_{k - 1} ave )}^{2}}{N * M}}\end{matrix}} \qquad [ Expression 6 ]$$

In addition, Ekave is an average of the element data Ek (i, j) in all items of the elastic frame data Frk. Only when Ck is higher than the reference value Cstd set in advance, Frk and Frk−1 are stored in the Cine memory 48 or the like. In addition, it is also possible to calculate a correlation between a pair of frames, which are adjacent to each other in time series, of the displacement frame data and to store a tomographic image and an elastic image in the Cine memory 48 or the recording medium 50, such as a DVD, only when the correlation is larger than the threshold value set in advance.

In addition, although a correlation between a pair of elastic frame data items adjacent to each other is calculated in the above, an examiner may select a frame, which is considered to be optimal, from the elastic frame data stored in advance in the Cine memory 48 or the recording medium 50, such as a DVD, and a correlation between the selected elastic frame data and elastic frame data acquired in real time may be calculated to evaluate the value of the image.

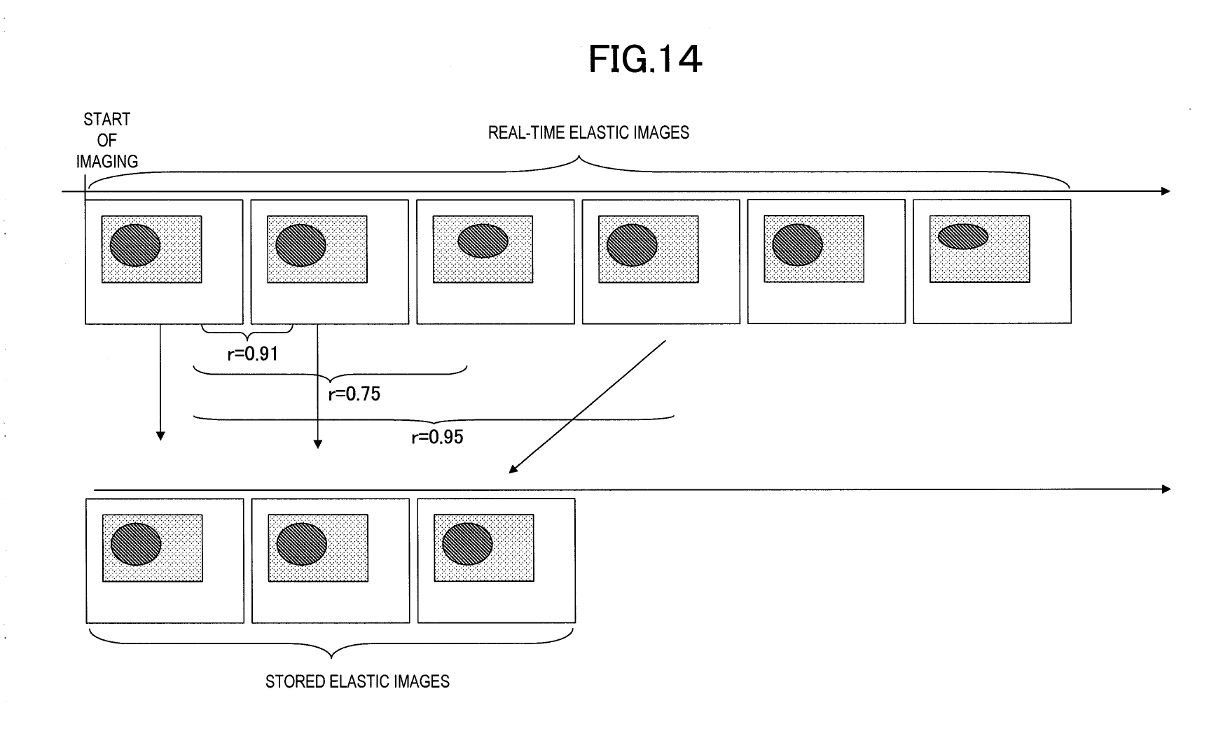

Alternatively, for example, as shown in FIG. 14, it is also possible to set elastic frame data after freeze ON or OFF or immediately after the start of imaging as reference elastic frame data, calculate a correlation between the reference elastic frame data and the elastic frame data acquired in real time, and store a tomographic image and an elastic image in the Cine memory 48 or the recording medium 50, such as a DVD, only when the correlation is larger than the threshold value set in advance.

### Sixth Example

Next, a sixth example will be described. In this example, average displacement frame data or average elastic frame data is calculated by averaging at least either displacement of plural corresponding measurement points of plural displacement frame data items belonging to a predetermined time section or elastic information of plural corresponding measurement points of plural elastic frame data items belonging to a predetermined time section, a correlation between the average displacement frame data or the average elastic frame data and the plural displacement frame data items or the plural elastic frame data items belonging to the predetermined time section is calculated, and it is evaluated that the pressure state is appropriate when the correlation is larger than the threshold value set in advance.

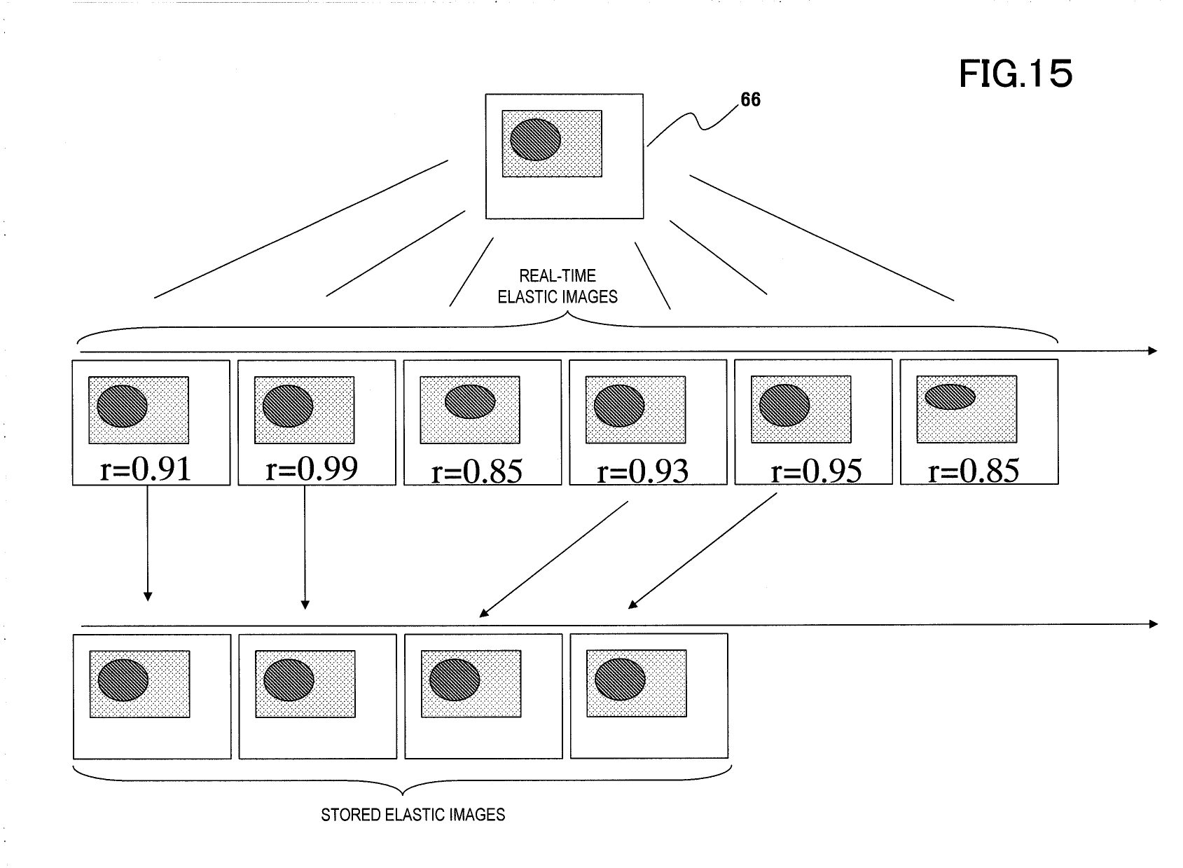

That is, elements of elastic frame data belonging to a certain designated time section are equalized and average elastic frame data 66 having these as elements is generated as shown in FIG. 15, for example. Then, it is also possible to calculate a correlation between the average elastic frame data 66 and each element of the elastic frame data and to store a tomographic image and an elastic image, which are based on elastic frame data with a high correlation and tomographic frame data corresponding to the elastic frame data, selectively in the Cine memory 48 or the recording medium 50, such as a DVD.

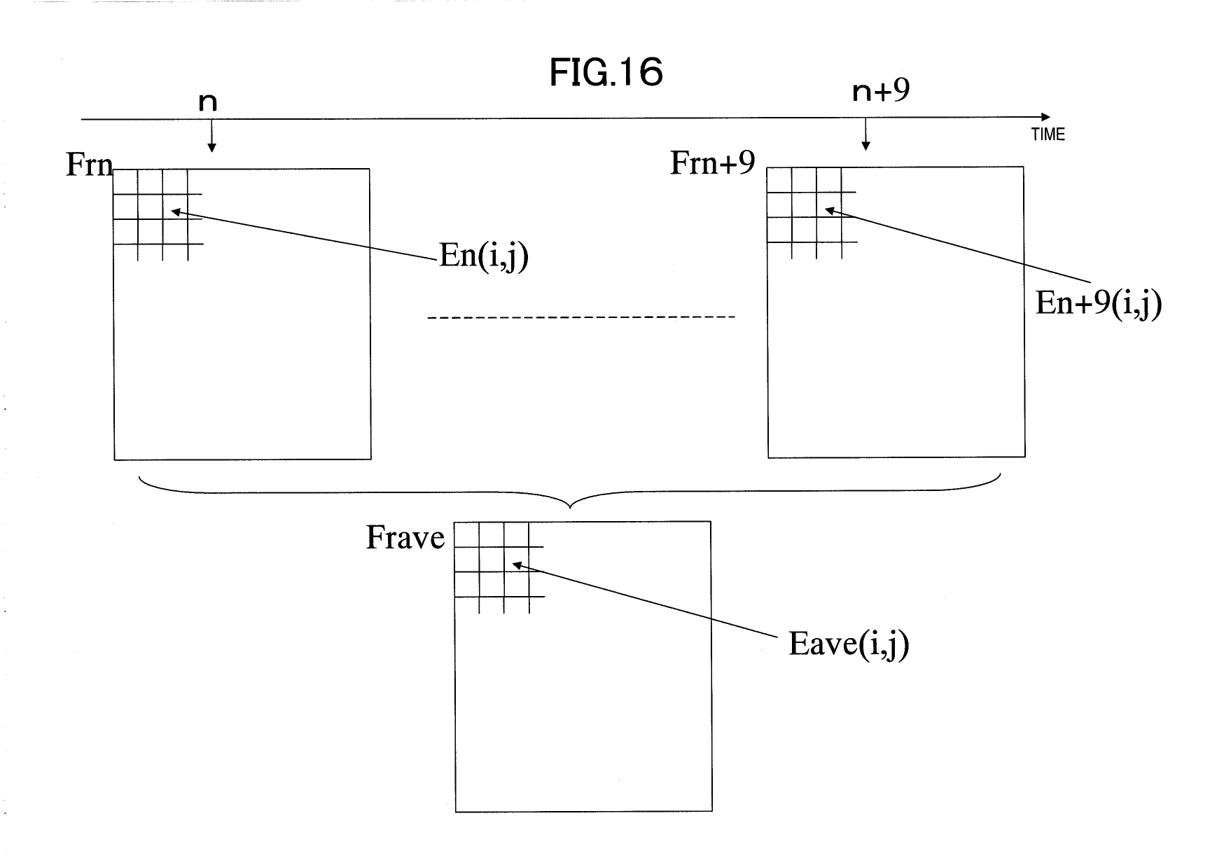

More specifically, as shown in FIG. 16, assuming that a certain time section is 10, for example, the pressure evaluating section 52 performs the following operation first to create average elastic frame data Frave having Em(i, j) (0≦i≦N, 0≦j≦M) as elements.

$$Em ( i , j ) = ( {\sum }_{l = n}^{n + 9} El ( i , j ) ) / 10 \qquad [ Expression 7 ]$$

Then, a correlation CI between Em(i, j) and El(i, j) is calculated by the following Expression 8.

$$Cl = \frac{\frac{\begin{matrix}{\sum }_{i = 0}^{N} {\sum }_{j = 0}^{M} ( {E}_{l} ( i , j ) - {E}_{l} ave ) \\ ( {E}_{m} ( i , j ) - {E}_{m} ave )\end{matrix}}{N * M}}{\begin{matrix}\sqrt{\frac{{\sum }_{i = 0}^{N} {\sum }_{j = 0}^{M} {( {E}_{l} ( i , j ) - {E}_{l} ave )}^{2}}{N * M}} \\ \sqrt{\frac{{\sum }_{i = 0}^{N} {\sum }_{j = 0}^{M} {( {E}_{m} ( i , j ) - {E}_{m} ave )}^{2}}{N * M}}\end{matrix}} \qquad [ Expression 8 ]$$

In addition, Elave is an average of the element data El (i, j) in all items of the elastic frame data Frl. Similarly, Emave is an average of the element data Em(i, j) in all items of the elastic frame data Frave. Only when the correlation is higher than the predetermined reference value Cstd, a tomographic image and an elastic image are stored in the Cine memory 48 or the recording medium 50, such as a DVD. In addition, the predetermined reference value Cstd correlated with a certain time section may be arbitrarily selected by external control of an examiner using a keyboard.

### Seventh Example

Next, a seventh example will be described. In this example, it is evaluated that the pressure state is not appropriate when a state where the pressure of the ultrasonic probe 12 on the ultrasonic wave transmission/reception surface is smaller than the threshold value set in advance continues for a predetermined time or more. In addition, it is evaluated that the pressure state is not appropriate when a state where at least one of the average value of displacement of plural measurement points of displacement frame data and the average value of elastic information of plural measurement points of elastic frame data is smaller than the threshold value set in advance continues for a predetermined time or more.

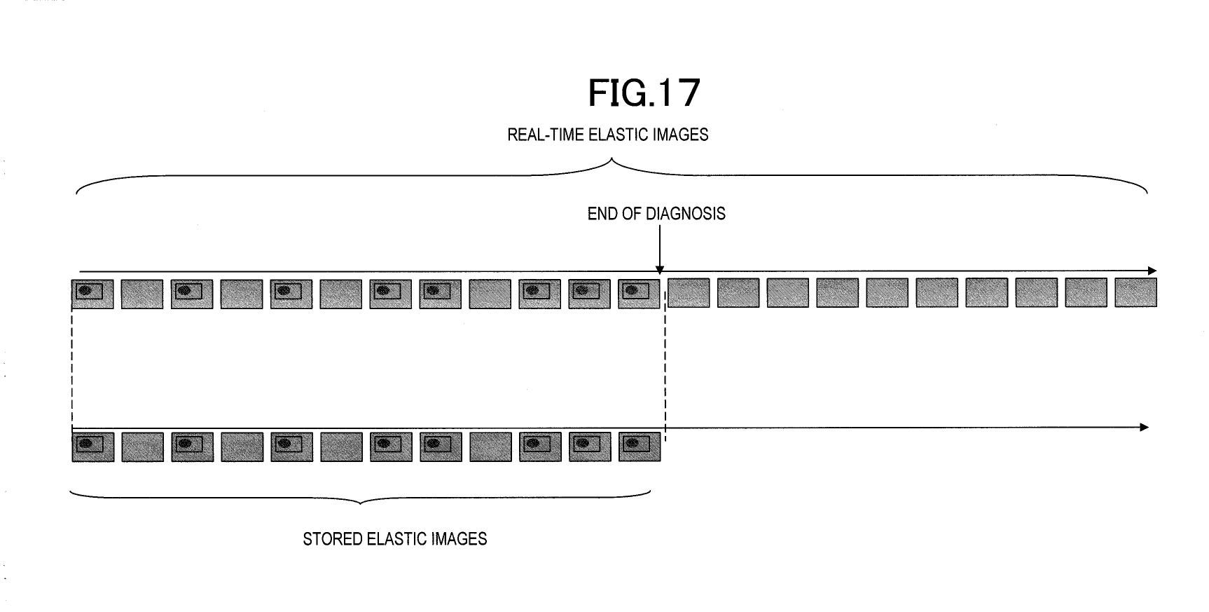

That is, in the examination process of actual ultrasonic diagnosis, the examiner does other work while continuing ultrasonic imaging even after the diagnosis ends and accordingly, a diagnostic image and an image useless for diagnosis may be stored in the Cine memory 48 or the like. Therefore, in this example, as shown in FIG. 17, a time for which the pressure evaluating section 52 does not perform elastic image diagnosis is detected on the basis of the displacement output from the displacement measuring section 30, the elastic modulus and distortion output from the elastic information calculating section 32, and the pressure output from the pressure measuring section 46, and corresponding images are not stored automatically in the Cine memory 48 or the like while elastic image diagnosis is not being performed.

More specifically, when diagnosis is not performed, the ultrasonic probe 12 is usually fixed to the holder without being in contact with the object 10. For this reason, the probe surface pressure is equal to 0 and there is no variation either. Accordingly, as an example of a method of detecting a time for which diagnosis is not performed, when the pressure acquired from the pressure measuring section 46 is equal to 0 and there is no change for a predetermined period, the pressure evaluating section 52 sets a pressure determination flag to 0 so that a tomographic image and an elastic image in the predetermined period are not stored in the Cine memory 48 or the recording medium 50, such as a DVD.

Alternatively, as a method of detecting a time for which diagnosis is not performed, it is also possible to use “elastic modulus is 0 for all elements in a frame”, “distortion is 0 for all elements in a frame”, and “displacement is 0 for all elements in a frame”.

### Eighth Example

Next, an eighth example will be described. In this example, it is evaluated whether or not the pressure state is appropriate using each method described above. Also, only when the ratio of elastic images in the appropriate pressure state to elastic images belonging to a predetermined time section is larger than the threshold value set in advance, the elastic images in the appropriate pressure state are stored in a memory. Alternatively, when reproducing elastic images stored in the memory, the elastic images in the appropriate pressure state are selectively read and reproduced.

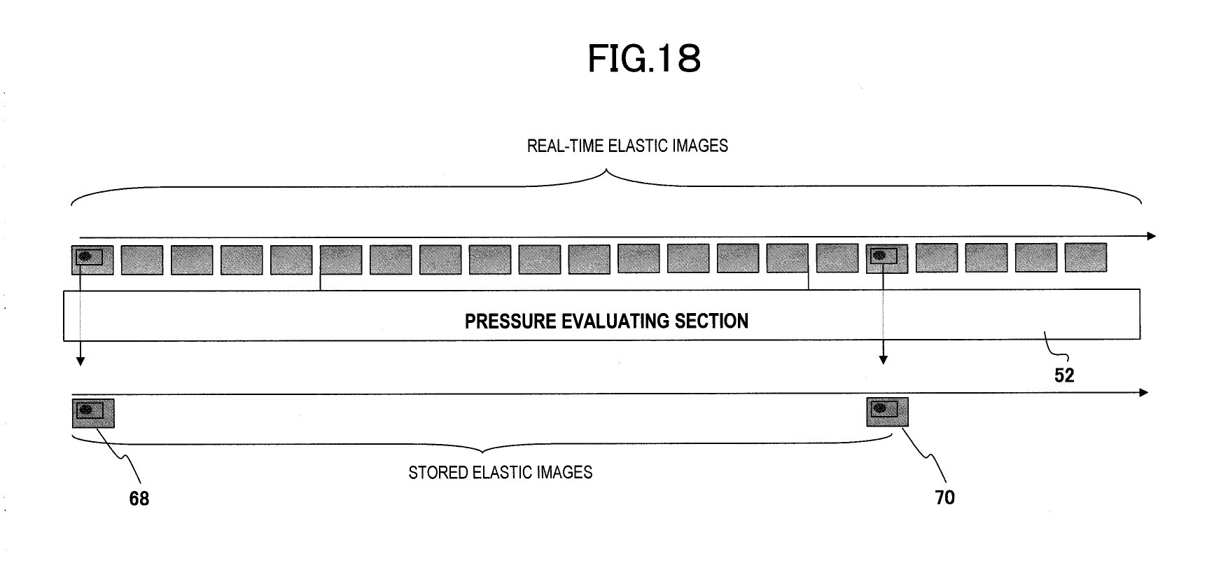

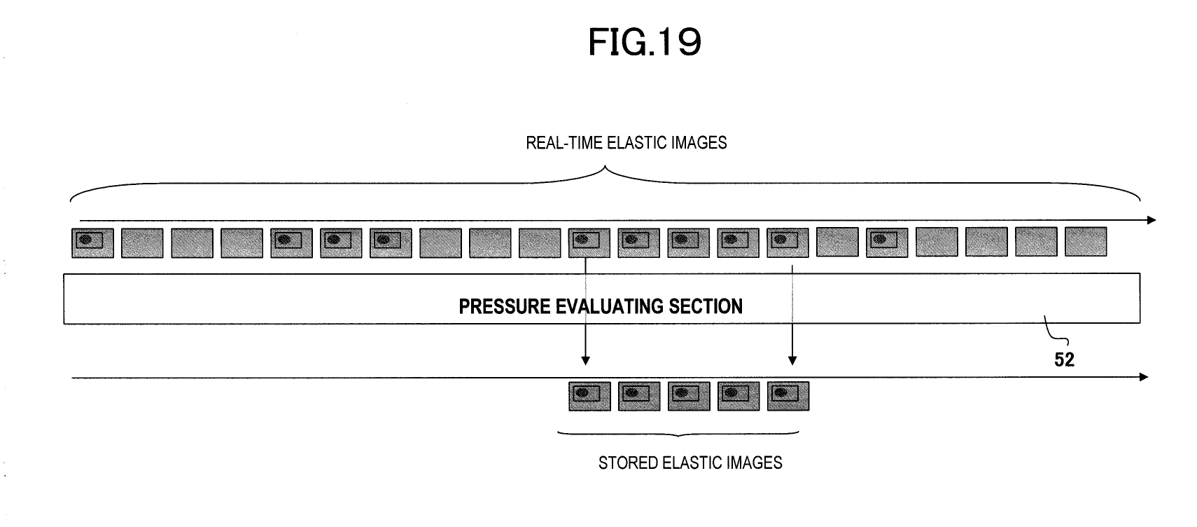

That is, when it is determined whether or not the pressure state is appropriate using the method described until now and corresponding images are stored in the Cine memory 48 or the like, continuity between elastic images 68 and 70 stored in the Cine memory 48 or the like may disappear if a frame, in which the pressure state is not appropriate, continues as shown in FIG. 18. This may lead to an unnatural image at the time of reproduction. Therefore, in this example, the number of frames with an appropriate pressure state is counted and a tomographic image and an elastic image are stored and reproduced only when, for example, 5 or more frames with an appropriate pressure state continue, as shown in FIG. 19. Moreover, as an example of determination regarding continuity, the rate of the number of frames with an appropriate pressure state may be checked every ten frames and a tomographic image and an elastic image with an appropriate pressure state may be stored and reproduced if the rate is equal to or higher than 80 percent or more, for example.

In addition, although the ultrasonic diagnostic apparatus and the elastic image storage/reproduction method have been mainly described in the above examples, the present invention is not limited to these. For example, the present invention may also be applied as an elastic image storage/reproduction program which can be installed in an ultrasonic diagnostic apparatus or a computer, such as a PC, and be executed. The elastic image storage/reproduction program is configured to include: evaluating whether or not a pressure state of an object is appropriate on the basis of at least one of displacement frame data of tissue of a sectional part generated on the basis of a pair of RF signal frame data items which are based on reflected echo signals measured in advance by an ultrasonic probe and whose acquisition times are different, elastic frame data indicating the hardness or softness of the tissue of the sectional part generated on the basis of the displacement frame data, and pressure of the ultrasonic probe on the ultrasonic wave transmission/reception surface; and storing an elastic image in the appropriate pressure state in a memory selectively or reading and reproducing an elastic image in the appropriate pressure state selectively when reproducing elastic images stored in the memory.

According to this, since elastic images generated in a state where the object 10 is appropriately pressed are selectively stored in a memory, an elastic image suitable for diagnosis is selectively reproduced when an examiner performs diagnosis by reproducing the elastic image stored in the memory. Alternatively, when reproducing elastic images stored in the memory, an elastic image in the appropriate pressure state is read and reproduced selectively. Therefore, since it does not take time for frame feeding or skipping when elastic images which are not suitable for diagnosis continue, good usability for the examiner is realized and it is possible to shorten the diagnostic time by improving the diagnostic efficiency.

### REFERENCE SIGNS LIST

10: object
12: ultrasonic probe
18: phasing addition section
30: displacement measuring section
32: elastic information calculating section
34: elastic image configuration section
46: pressure measuring section
48: Cine memory
50: recording medium such as a DVD
52: pressure evaluating section
60: pressure graph
62: pressure graph
64: optimal pressure graph
66: average elastic frame data
100: ultrasonic diagnostic apparatus

1. An ultrasonic diagnostic apparatus comprising:
an ultrasonic probe which transmits and receives an ultrasonic wave to and from an object;
a phasing addition means that generates RF signal frame data of a sectional part of the object on the basis of a reflected echo signal measured by the ultrasonic probe;
a displacement measurement means that generates displacement frame data by measuring displacement of tissue of the sectional part on the basis of a pair of RF signal frame data acquired at different times;
an elastic information calculation means that generates elastic frame data by calculating elastic information, which indicates hardness or softness of tissue of the sectional part, on the basis of the generated displacement frame data;
an elastic image configuration means that generates an elastic image on the basis of the elastic frame data;
an image display device which displays the elastic image;
a memory in which the elastic image is stored; and
a pressure evaluating section which evaluates whether or not a pressure state of the object is appropriate on the basis of at least one of the displacement frame data of the tissue of the sectional part, the elastic frame data, and pressure of the ultrasonic probe on an ultrasonic wave transmission/reception surface,
wherein an elastic image in an appropriate pressure state is stored in the memory selectively.

2. The ultrasonic diagnostic apparatus according to claim 1,
wherein an elastic image in the appropriate pressure state is read and reproduced selectively when elastic images stored in the memory are reproduced.

3. The ultrasonic diagnostic apparatus according to claim 1,
wherein the pressure evaluating section evaluates that the pressure state is appropriate when an absolute value of at least one of an average value of displacement of a plurality of measurement points of the displacement frame data and an average value of elastic information of a plurality of measurement points of the elastic frame data is larger than a threshold value set in advance.

4. The ultrasonic diagnostic apparatus according to claim 1,
wherein the pressure evaluating section evaluates that the pressure state is appropriate when an absolute value of a rate of change of the pressure of the ultrasonic probe on the ultrasonic wave transmission/reception surface is larger than a threshold value set in advance.

5. The ultrasonic diagnostic apparatus according to claim 1,
wherein the pressure evaluating section evaluates that the pressure state is appropriate when at least one of a variance or deviation of displacement of a plurality of measurement points of the displacement frame data and a variance or deviation of elastic information of a plurality of measurement points of the elastic frame data is smaller than a threshold value set in advance.

6. The ultrasonic diagnostic apparatus according to claim 1,
wherein the pressure evaluating section evaluates that the pressure state is appropriate when a variance or deviation of pressure at a plurality of places including at least both ends of the ultrasonic wave transmission/reception surface of the ultrasonic probe in a beam line direction is smaller than a threshold value set in advance.

7. The ultrasonic diagnostic apparatus according to claim 1,
wherein the pressure evaluating section evaluates that the pressure state is appropriate when at least one of a displacement gradient of a plurality of measurement points of the displacement frame data in a beam line direction of the ultrasonic probe, a variance of displacement of the plurality of measurement points of the displacement frame data in the beam line direction, and a deviation of displacement of the plurality of measurement points of the displacement frame data in the beam line direction is smaller than a threshold value set in advance.

8. The ultrasonic diagnostic apparatus according to claim 1,
wherein the pressure evaluating section calculates a correlation between at least one of a graph showing a temporal change in the pressure on the ultrasonic wave transmission/reception surface, a graph showing a temporal change in displacement of tissue of the sectional part, and a graph showing a temporal change in elastic information of tissue of the sectional part and a corresponding one of an optimal pressure graph, an optimal displacement graph, and an optimal elastic information graph, which are set in advance for the graphs, and evaluates that the pressure state is appropriate when the correlation is larger than a threshold value set in advance.

9. The ultrasonic diagnostic apparatus according to claim 1,
wherein the pressure evaluating section calculates a correlation between a pair of frames, which are adjacent to each other in time series, of either the displacement frame data or the elastic frame data and evaluates that the pressure state is appropriate when the correlation is larger than a threshold value set in advance.

10. The ultrasonic diagnostic apparatus according to claim 1,
wherein the pressure evaluating section calculates average displacement frame data or average elastic frame data by averaging at least either displacement of a plurality of corresponding measurement points of the plurality of displacement frame data items belonging to a predetermined time section or elastic information of a plurality of corresponding measurement points of the plurality of elastic frame data items belonging to a predetermined time section, calculates a correlation between the average displacement frame data or the average elastic frame data and the plurality of displacement frame data items or the plurality of elastic frame data items belonging to the predetermined time section, and evaluates that the pressure state is appropriate when the correlation is larger than a threshold value set in advance.

11. The ultrasonic diagnostic apparatus according to claim 1,
wherein the pressure evaluating section evaluates that the pressure state is not appropriate when a state where the pressure of the ultrasonic probe on the ultrasonic wave transmission/reception surface is smaller than a threshold value set in advance continues for a predetermined time or more.

12. The ultrasonic diagnostic apparatus according to claim 1,
wherein the pressure evaluating section evaluates that the pressure state is not appropriate when a state where at least one of an average value of displacement of a plurality of measurement points of the displacement frame data and an average value of elastic information of a plurality of measurement points of the elastic frame data is smaller than a threshold value set in advance continues for a predetermined time or more.

13. The ultrasonic diagnostic apparatus according to claim 1,
wherein the pressure evaluating section evaluates whether or not the pressure state is appropriate and also stores an elastic image in the appropriate pressure state in the memory selectively only when a ratio of elastic images in the appropriate pressure state to elastic images belonging to a predetermined time section is larger than a threshold value set in advance.

14. An elastic image storage/reproduction method comprising;
generating RF signal frame data on the basis of a reflected echo signal measured by an ultrasonic probe;
generating displacement frame data by calculating displacement of tissue of the sectional part on the basis of a pair of RF signal frame data items acquired at different times;
generating elastic frame data by calculating elastic information, which indicates hardness or softness of tissue of the sectional part, on the basis of the displacement frame data;
generating an elastic image on the basis of the elastic frame data;
evaluating whether or not a pressure state of than object is appropriate on the basis of at least one of the displacement frame data, the elastic frame data, and pressure of the ultrasonic probe on an ultrasonic wave transmission/reception surface; and
storing an elastic image in an appropriate pressure state in a memory selectively or reading and reproducing an elastic image in the appropriate pressure state selectively when reproducing elastic images stored in the memory.

15. An elastic image storage/reproduction program comprising steps of:
evaluating whether or not a pressure state of an object is appropriate on the basis of at least one of displacement frame data of tissue of sectional part generated on the basis of a pair of RF signal frame data items which are based on reflected echo signals measured by an ultrasonic probe and whose acquisition times are different, elastic frame data indicating hardness or softness of the tissue of the sectional part generated on the basis of the displacement frame data, and pressure of the ultrasonic probe on an ultrasonic wave transmission/reception surface; and
storing an elastic image in an appropriate pressure state in a memory selectively or reading and reproducing an elastic image in the appropriate pressure state selectively when reproducing elastic images stored in the memory.
